(19) United States
(12) Patent Application Publication
(10) Pub. No.: US 2004/0166485 A1
(43) Pub. Date: Aug. 26, 2004

(54) Materials and methods relating to cholesterol biosynthesis enzymes

(75) Inventors: Gail E. Herman, Columbus, OH (US); Richard I. Kelley, Strafford, PA (US); Dorothy K. Grange, St. Louis, MO (US)

Correspondence Address: MARSHALL, GERSTEIN & BORUN LLP, 6300 SEARS TOWER, 233 S. WACKER DRIVE, CHICAGO, IL, 60606, US

(73) Assignee: CHILDREN'S HOSPITAL INC, Columbus, OH (US)

(21) Appl. No.: 10/684,978

(22) Filed: Oct. 14, 2003

Related U.S. Application Data
(63) Continuation of application No. 09/946,406, filed on Sep. 5, 2001.
Application No. 09/946,406 is a continuation of application No. 09/588,976, filed on Jun. 1, 2000, now abandoned.
(60) Provisional application No. 60/137,020, filed on Jun. 1, 1999.

(51) Int. Cl. C12Q 1/00; G01N 33/92

(52) U.S. Cl. 435/4; 436/71

(57) ABSTRACT

The present invention relates to the identification of the NSDHL gene product as a 3β-HSD participating in the conversion of 4,4-dimethylcholest-8(9)-en-3β-ol to cholest-8(9)-en-3β-ol in the cholesterol and vitamin D biosynthetic pathway. Based upon this function the present invention contemplates methods for manipulating the biosynthetic pathway at the step of involvement of NSDHL to increase or decrease the levels of cholesterol and/or vitamin D (or downstream products such as steroids) produced by a cell. Also contemplated are methods for manipulating the accumulation of intermediate compounds upstream of the step of NSDHL in the pathway. Diagnostic methods involving identification of mutations in the genes encoding enzymes involved in the conversion of 4,4-dimethylcholest-8(9)-en-3β-ol to cholest-8(9)-en-3β-ol are also provided, as well as diagnostic methods involving detection of abnormal accumulation of sterol intermediates prior to generation of choles-8(9)-en-3β-ol in the pathway.

This is a continuation-in-part of U.S. Patent Application Serial No. 60/137,020 filed Jun. 1, 1999.

### FIELD OF THE INVENTION

The present invention generally relates to 3β-hydroxysteroid dehydrogenase (3β-HSD) enzymes that participate in cholesterol and vitamin D biosynthesis. More particularly, the invention relates to the 3β-HSD enzyme termed NSDHL and to manipulation of the chemical reaction(s) in which it participates.

### BACKGROUND

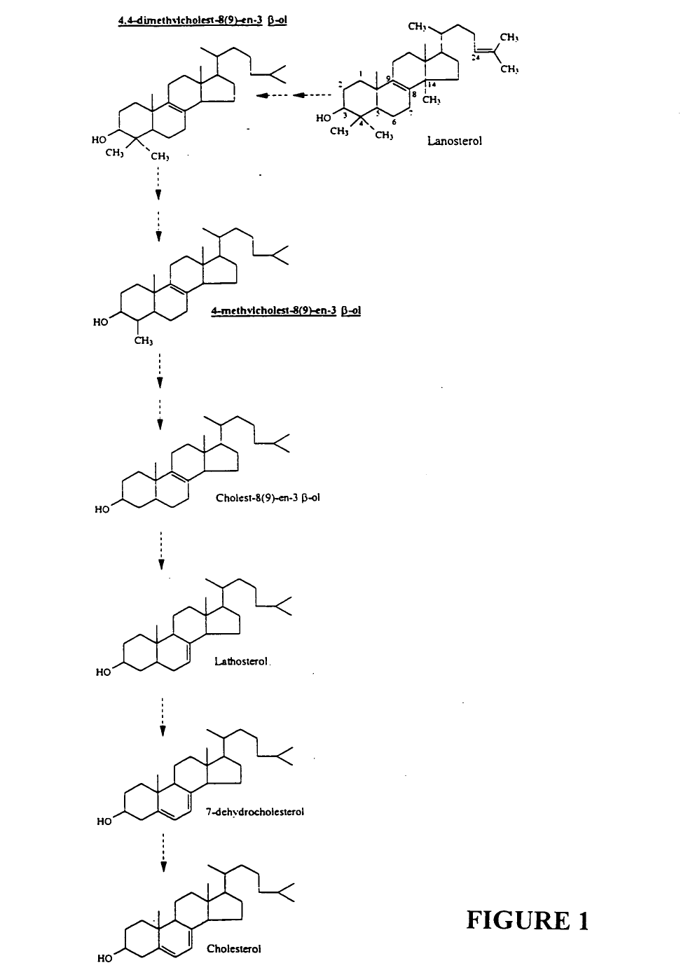

Cholesterol is a key component of cell membranes and is the immediate precursor for the synthesis of all known steroid hormones and bile acids. Vitamin D is important in calcium homeostasis and bone formation. Cholesterol and vitamin D can be synthesized in a multistep pathway from the sterol precursor lanosterol. As illustrated in FIG. 1, lanosterol is typically first converted to 4,4-dimethylcholest-8(9)-en-3β-ol. Next, 4,4-dimethylcholest-8(9)-en-3β-ol is converted to 4-methylcholest-8(9)-en-3β-ol and then to cholest-8(9)-en-3β-ol by sequential removal of the two C-4 methyl groups from the sterol backbone. Cholest-8(9)-en-3β-ol is, in turn, converted to lathosterol (cholest-7-en-3β-ol). 7-dehydrocholesterol is then generated from lathosterol and is the immediate major precursor of both cholesterol and vitamin D. Intermediates preceding lanosterol in the cholesterol biosynthetic pathway also serve as precursors for the synthesis of non-sterol isoprenes such as isopentenyl-tRNAs, dolichol, ubiquinone, and haem A. The isopentenyl groups in tRNAs stabilize codon-anticodon interaction contributing to fidelity of protein synthesis from mRNA. Dolichol is required for the synthesis of glycoproteins. Ubiquinone and haem A are important components of the mitochondrial respiratory chain. While some genes encoding enzymes participating in the mammalian biosynthetic pathway have been previously characterized, other genes remain unknown.

Various disease states have been linked to abnormalities in cholesterol biosynthesis. For example, mevalonic aciduria was the first disorder of cholesterol synthesis to be recognized and is caused by a defect in mevalonate kinase, an enzyme participating in early steps of the pathway. Patients experience developmental delay, failure to thrive, hypotonia, ataxia, hepatosplenomegaly, cataracts, lymphadenopathy, anaemia, myopathy, and enteropathy with fat malabsorption.

Additionally, defects in later steps of the pathway in the conversion of lanosterol to cholesterol have been described in a single infant with “desmosterolosis” in Clayton, Arch. Dis. Child., 78: 185-189 (1998) and in patients with Smith-Lemli-Opitz syndrome (SLOS) in Kelley, Am. J. Hum. Genet., 63: 322-326 (1998). The desmosterolosis afflicted infant had a cleft palate, ambiguous genitalia, short limbs, short and unrotated intestines and bilateral renal hypoplasia. SLOS is characterized by mental retardation, microcephaly, failure to thrive, cataracts, cleft palate, postaxial polydactyly and/or syndactyly of the second and third toes, congenital heart disease, genital abnormalities, photosensitivity, and, occasionally, chondrodysplasia punctata. Affected patients accumulate 7-dehydrocholesterol and mutations in the 3β-hydroxysteriod-Δ⁷-reductase gene have recently been identified in numerous SLOS probands. See, e.g., Wassif et al., Am. J. Hum. Genet., 63: 66-62 (1998). Even more recently, Kelley et al., Am. J. Med. Gen., 83: 213-219 (1999) reported that patients with X-linked dominant Conradi-Hünermann-Happle syndrome and nonspecific lethal chondrodysplasia punctata exhibited abnormally increased levels of 8-dehydrocholesterol and cholest-8(9)-en-3β-ol.

Other disease states have been suggested to involve 3β-HSD abnormalities but the biosynthetic pathway in which those enzymes participated had not previously been demonstrated. For example, Herman et al. [presenting at the 12^{th} International Mouse Genome Conference, Garmisch, Germany (September 1998) and at the American Society of Human Genetics Meeting Late Breaking Plenary Session, Denver, Colo. (October 1998)] described an X-linked mouse mutation bare patches (Bpa) occurring in a gene, the Nsdhl gene putatively encoding a 3β-HSD, as being a murine model of the human X-linked dominant disorder chondrodysplasia punctata (CDPX2). The human and murine phenotypes of the two disorders are very similar and include hyperkeratotic skin eruptions and hair loss, a skeletal dysplasia characterized by abnormal epiphyseal calcifications (chondrodysplasia punctata), and frequent cataracts and/or microophthalmia. A human genomic DNA containing a gene (NSDHL) corresponding to the murine Nsdhl gene was disclosed in Genbank Accession No. U82671. The human NSDHL gene has been mapped on the X chromosome at Xq28 and comprises eight exons. At the time of the Herman et al. presentations, the substrate of the putative enzyme encoded by the human NSDHL and murine Nsdhl genes was not known. However, Gachotte et al., Proc. Natl. Acad. Sci. USA, 95: 13794-13799 (1998) reported that the S. cerevisiae homolog of Nsdhl functions as a C-3 sterol dehydrogenase (C-4 decarboxylase) in the synthesis of ergosterol, the major sterol of yeast and Kelley et al. (1999), supra suggested that Nsdhl gene product may have a role in cholesterol biosynthesis in 4-demethylation of lanosterol.

There thus continues to exist in the art a need for the identification of genes encoding enzymes that participate in the biosynthetic pathway(s) for cholesterol and vitamin D as well as for the identification of disease states resulting from mutations in those genes or abnormalities in their expression.

### BRIEF SUMMARY OF THE INVENTION

The present invention relates to the identification of the NSDHL gene product as a 3β-HSD involved in the conversion of 4,4-dimethylcholest-8(9)-en-3β-ol to cholest-8(9)-en-3β-ol in the cholesterol and vitamin D biosynthetic pathway.

Based upon this function, the present invention contemplates methods for manipulating the biosynthetic pathway at the step of involvement of NSDHL to increase or decrease the levels of cholesterol and/or vitamin D (or downstream products such as steroids) produced by a cell. Also contemplated are methods for manipulating (increasing or decreasing) the accumulation of intermediate compounds upstream of the step of involvement of NSDHL in the pathway. These methods of manipulation are useful, for example, in in vitro cell culture methods to produce steroids for which cholesterol is a precursor and in in vivo treatments for disease states involving the biosynthetic pathway.

The present invention contemplates that various disease states including bone, skin and eye disorders are associated with mutations in the NSDHL gene and other genes encoding proteins involved in the conversion of 4,4-dimethylcholest-8(9)-en-3β-ol to cholest-8(9)-en-3β-ol. Thus, detection of mutations in these genes is useful in the initial diagnosis of the disease states and in evaluating treatment options.

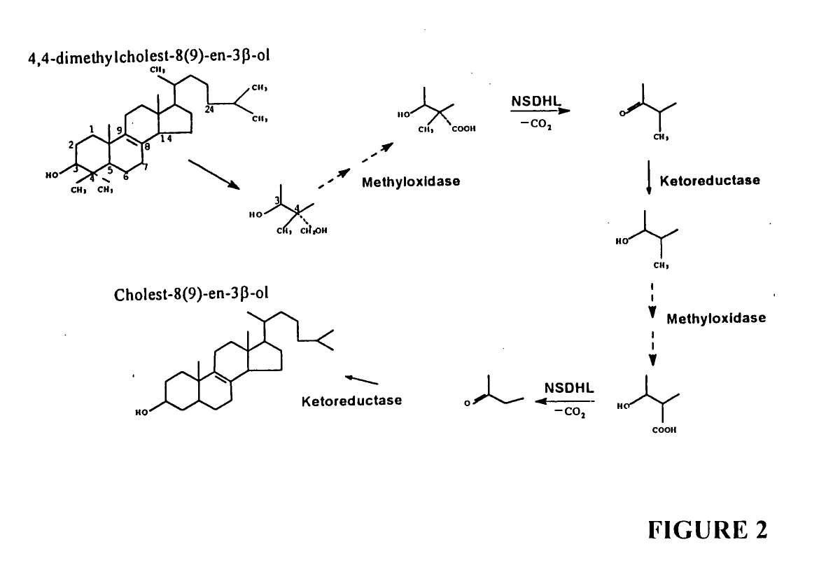

Embodiments of the invention are methods for diagnosing disease states associated with mutations in the NSDHL gene or the other genes encoding proteins involved in the same step of the biosynthetic pathway, that is the step in which 4,4-dimethylcholest-8(9)-en-3β-ol is converted to cholest-8(9)-en-3β-ol. It is contemplated that the step of removal of the two C-4 methyl groups requires the sequential actions of a C-4 sterol methyloxidase, the NSDHL gene product (a 3β-HSD) and a 3-keto reductase (i.e., the sterol-4-demethylase complex). See FIG. 2. In addition, regulatory or other accessory proteins may be required. A cDNA sequence encoding a human C-4 sterol methyloxidase is deposited under GenBank U60205 (SEQ ID NO: 5 herein) and was identified based on homology to yeast ERG25 [Li and Kaplan, J. Biol. Chem., 271: 16927-16933 (1996)].

In the diagnostic methods of the invention, polynucleotides obtained from a patient are analyzed for mutations (i.e., nucleotide differences from wild type) using primers or probes based on the NSDHL polynucleotide sequence set out in SEQ ID NO: 1 or the C-4 sterol methyloxidase sequence set out in SEQ ID NO: 5 using standard polynucleotide sequencing, amplification and/or hybridization techniques. Examples of intronic primer pairs for routine sequencing of and mutation detection in the 5′ noncoding region and eight exons of the human NSDHL gene are:

| | |
|---|---|
| 5′ noncoding | |
| 5′ AAAGACTGGTGCGCTAAAGC 3′ | (SEQ ID NO: 7) |
| 5′ CGAGCTTCCTCCACCAAGAG 3′ | (SEQ ID NO: 8) |
| Exon 1 (ending at nucleotide 151 of SEQ ID NO: 1) | |
| 5′ CCCCGTCTTTATTGGGCAAG 3′ | (SEQ ID NO: 9) |
| 5′ ACTGCCCAGTCGCTGACACAG 3′ | (SEQ ID NO: 10) |
| Exon 2 (corresponding to nucleotides 152 through 302 of SEQ ID NO: 1) | |
| 5′ GGCATCTGCCCAAAACACTAAC 3′ | (SEQ ID NO: 11) |
| 5′ CCACAGGTAAATAGTATCAGCC 3′ | (SEQ ID NO: 12) |
| Exon 3 (corresponding to nucleotides 303 through 461 of SEQ ID NO: 1) | |
| 5′ TTCCAGTCCTCACTACCCTG 3′ | (SEQ ID NO: 13) |
| 5′ AGTATCGTGGTTTCCCTTCG 3′ | (SEQ ID NO: 14) |
| Exon 4 (corresponding to nucleotides 462 through 608 of SEQ ID NO: 1) | |
| 5′ TGCCATTGACCTGTCAAAGC 3′ | (SEQ ID NO: 15) |
| 5′ CCCTTAGAAAGGGCCATCAC 3′ | (SEQ ID NO: 16) |
| Exon 5 (corresponding to nucleotides 609 through 737 of SEQ ID NO: 1) | |
| 5′ GGATCATGCACTGTTTGAATTG 3′ | (SEQ ID NO: 17) |
| 5′ GGATTCTAAACCCTTCAGTC 3′ | (SEQ ID NO: 18) |
| Exon 6 (corresponding to nucleotides 738 through 880 of SEQ ID NO: 1) | |
| 5′ CTAGGAATTTGCAATGGACG 3′ | (SEQ ID NO: 19) |
| 5′ TGAATGCGAGCATGGACCAG 3′ | (SEQ ID NO: 20) |
| Exon 7 (corresponding to nucleotides 881 through 983 of SEQ ID NO: 1) | |
| 5′ AAGACTTGGGAGTGGCCCTG 3′ | (SEQ ID NO: 21) |
| 5′ AGGCAAGGAGAAGAAACCCG 3′ | (SEQ ID NO: 22) |
| Exon 8 (beginning at nucleotide 984 of SEQ ID NO: 1) | |
| 5′ TTCAACTTTGGGCAGGTGGG 3′ | (SEQ ID NO: 23) |
| 5′ CTCCATAGCATCATCCATGG 3′ | (SEQ ID NO: 24) |
| and | |
| 5′ CATTCCACTACTACAGCTGC 3′ | (SEQ ID NO: 25) |
| 5′ TGTATAAACCAGAAGAGGGG 3′. | (SEQ ID NO: 26) |

Techniques contemplated by the invention include, but are not limited to, well-known techniques such as polymerase chain reaction techniques, single-strand conformation polymorphism analysis (SSCP) [Orita et al., Proc Natl. Acad. Sci. USA, 86: 2766-2770 (1989)]; heteroduplex analysis [White et al., Genomics, 12: 301-306 (1992)]; denaturing gradient gel electrophoresis analysis [Fischer et al., Proc. Natl. Acad. Sci. USA, 80: 1579-1583 (1983); and Riesner et al., Electrophoresis, 10: 377-389 (1989)]; DNA sequencing; RNase cleavage [Myers et al., Science, 230: 1242-1246 (1985)]; chemical cleavage of mismatch techniques [Rowley et al., Genomics, 30: 574-582 (1995); and Roberts et al., Nucl. Acids Res., 25: 3377-3378 (1997)]; restriction fragment length polymorphism analysis; single nucleotide primer extension analysis [Shumaker et al., Hum. Mutat., 7: 346-354 (1996); and Pastinen et al., Genome Res., 7: 606-614 (1997)]; 5′ nuclease assays [Pease et al., Proc. Natl. Acad. Sci. USA, 91:5022-5026 (1994)]; DNA Microchip analysis [Ramsay, G., Nature Biotechnology, 16: 40-48 (1999); and Chee et al., U.S. Pat. No. 5,837,832]; and ligase chain reaction [Whiteley et al., U.S. Pat. No. 5,521,065]. See generally, Schafer and Hawkins, Nature Biotechnologyy, 16: 33-39 (1998). All of the foregoing documents are hereby incorporated by reference in their entirety.

In one preferred embodiment the assaying involves sequencing of nucleic acid to determine nucleotide sequence thereof, using any available sequencing technique. [See, e.g., Sanger et al., Proc. Natl. Acad. Sci. (USA), 74: 5463-5467 (1977) (dideoxy chain termination method); Mirzabekov, TIBTECH, 12: 27-32 (1994) (sequencing by hybridization); Drmanac et al., Nature Biotechnology, 16: 54-58 (1998) and Science, 260: 1649-1652 (1993) (sequencing by hybridization); Kieleczawa et al., Science, 258: 1787-1791 (1992) (sequencing by primer walking); (Douglas et al., Biotechniques, 14: 824-828 (1993) (Direct sequencing of PCR products); and Akane et al., Biotechniques 16: 238-241 (1994); Maxam and Gilbert, Meth. Enzymol., 65: 499-560 (1977) (chemical termination sequencing)].

Alternatively, diagnostic methods of the invention involve detection in a patient's body fluid (e.g., serum, urine, semen, amniotic fluid, saliva, pleural fluid, peritoneal fluid, and cerebrospinal fluid) or cells (e.g., fibroblasts, keratinocytes, chondrocytes, liver tissue or osteoid tissue) of sterol intermediates or their metabolites prior to the step of generation of cholest-8(9)-en-3β-ol in the cholesterol/vitamin D biosynthetic pathway. Preferably detection is by gas chromotography and mass spectrometry (GC/MS) or related mass spectrometric techniques, and more preferably is by selected ion monitoring gas chromatography/mass-spectrometry (SIM-GC/MS) as described in Kelley, Clinica Chimica Acta, 236: 45-58 (1995) which is incorporated by reference herein. In the methods, a sample obtained from a patient is analzyed by SIM-GC/MS for accumulation of 4,4-dimethylcholest-8(9)-en-3β-ol, 4-methylcholest-8(9)-en-3β-ol (methylsterol-1) and/or 4-methylcholesta-8(9),24-dien-3β-ol (methylsterol-2), sterol intermediates generated prior to or during the NSDHL step of involvement in the biosynthetic pathway. Accumulation of these intermediates is indicative of an abnormality at this step in the pathway because only trace concentrations of the intermediates are detectable in normal individuals.

Preferred diagnostic methods contemplated by the invention are methods of diagnosis of CHILD syndrome (involving skin and bone), of skin disorders such as psoriasis and ichthyoses, of bone disorders such as osteoporosis and osteosclerosis, of eye disorders such as cataracts and microopthalmia and of arthritis.

In another embodiment, treatment of skin, bone and eye disorders is contemplated by the invention. Preferred indications are CHILD syndrome (involving skin and bone), skin disorders such as psoriasis and ichthyoses, bone disorders such as osteoporosis and osteosclerosis, eye disorders such as cataracts and microopthalmia, and arthritis. As noted above, manipulation of the biosynthetic pathway at the step of involvement of NSDHL may involve increasing or decreasing downstream products produced by a cell or may involve increasing or decreasing upstream products produced by a cell. This is because mutations in genes encoding proteins involved in the step alter the balance between upstream and downstream products. In disease states such as CHILD syndrome, psoriasis, ichthyoses, osteosclerosis, cataracts, micropthalmia and arthritis, it is contemplated that treatment according to the invention involves decreasing upstream products and increasing downstream products. Tissue abnormalities that arise in these disorders can be caused by adverse effects of intermediate sterols upstream of the step of involvement of NSDHL in the pathway (such as C29 sterols 4,4′-dimethylcholest-8-en-3β-ol and 4,4′-dimethylcholesta-8,24-dien-3β-ol) and/or deficiency in downstream products. In disease states such as osteoporosis it is contemplated that treatment involves increasing upstream products and decreasing downstream products in order to promote bone formation.

In one embodiment, agents to be used to therapeutically manipulate the biosynthetic pathway can be identified in assays based on the 3β-HSD activity of the NSDHL (SEQ ID NO: 2) or Nsdhl (SEQ ID NO: 4) enzyme products, or in assays based on the methyloxidase activity of C-4 sterol methyloxidase (SEQ ID NO: 6). The enzyme is made by standard recombinant techniques by expressing human NSDHL (SEQ ID NO: 1) or murine Nsdhl (SEQ ID NO: 3) or C-4 sterol methyloxidase cDNA in an appropriate host cell. Activity of the enzyme is measured in the presence and absence of a test agent and agents which increase or decrease the activity of the enzyme are identified. As one example, the active site of NSDHL enzyme (amino acids 92-96 of SEQ ID NO: 2) exhibits relatively low homology to other mammalian 3β-HSDs and is contemplated as a binding site for a agent that would inhibit the NSDHL enzyme.

In other embodiments, expression of the NSDHL gene or C-4 sterol methyloxidase gene is directly manipulated in vivo either locally (e.g., topically) or systemically, for example, using antisense or sense oligonucleotides comprising a single-stranded nucleic acid sequence (either RNA or DNA) capable of binding to target mRNA (sense) or DNA (antisense) sequences or using gene therapy.

Antisense or sense oligonucleotides, according to the present invention, comprise a fragment of DNA (such as that DNA set forth in SEQ ID NOs: 1 or 5). Such a fragment generally comprises at least about 14 nucleotides, preferably from about 17 to about 30 nucleotides. The ability to derive an antisense or a sense oligonucleotide, based upon a cDNA sequence encoding a given protein is described in, for example, Stein and Cohen (Cancer Res., 48:2659, 1988) and van der Krol et al. (BioTechiniques, 6:958, 1988). Binding of antisense or sense oligonucleotides to target nucleic acid sequences results in the formation of duplexes that block or inhibit protein expression by one of several means, including enhanced degradation of the mRNA by RNAseH, inhibition of splicing, premature termination of transcription or translation, or by other means. The antisense oligonucleotides thus may be used to block expression of proteins. Antisense or sense oligonucleotides further comprise oligonucleotides having modified sugar-phosphodiesterase backbones (or other sugar linkages) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequence. Other examples of sense or antisense oligonucleotides include those oligonucleotides which are covalently linked to organic moieties and other moieties that increases affinity of the oligonucleotide for a target nucleic acid sequence, such as poly-(L-lysine). Further still, intercalating agents, as ellipticine, and the alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, lipofection, CaPO₄-mediated DNA transfection, electroporation, or by using gene transfer vectors such as Epstein-Barr virus. Sense or antisense oligonucleotides also may be introduced into a cell containing the target nucleotide sequence by formation of a conjugate with a ligand binding molecule. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence formation of an oligonucleotide-lipid complex. The sense or antisense oligonucleotide-lipid complex is preferably dissociated within the cell by an endogenous lipase.

Mutations in the genes encoding proteins involved in the conversion of 4,4-dimethylcholest-8(9)-en-3β-ol to cholest-8(9)-en-3β-ol that result in reduction or loss of normal expression or function of the proteins are contemplated to underlie aforementioned disease states. The invention comprehends gene therapy to restore gene function to treat those disease states. For example, delivery of functional NSDHL or C-4 sterol methyloxidase genes to appropriate cells is effected ex vivo, in situ, or in vivo by use of vectors, and more particularly viral vectors (e.g., adenovirus, adeno-associated virus or retrovirus), or ex vivo by use of physical DNA transfer methods (e.g., liposomes or chemical treatments). Delivery may be systemic or local as appropriate. See, for example, Anderson, Nature, 392(6679 Suppl):25-30 (1998).

### DETAILED DESCRIPTION OF THE INVENTION

The invention is illustrated by the following examples wherein Example 1 describes the tissue distribution of Nsdhl mRNA, Example 2 describes abnormal sterol accumulation in Bpa mice, Example 3 reports mutations in the human NSDHL gene in patients with CHILD syndrome and Example 4 describes abnormal sterol accumulation in psoriasis patients.

### EXAMPLE 1

Previous Northern analyses with partial human NSDHL cDNA probes (called XAP104 and H105E3) demonstrated ubiquitous expression in adult tissues of a 1.5-2.0 kb transcript. See Levin et al., Genome Res., 6: 465-477 (1996) and Heiss et al., Genome Res., 6: 478-491 (1996).

Expression of wild type murine Nsdhl mRNA was examined and compared to the human tissue distribution results. Northern blots containing 4 μg of mRNA were prepared from cultured undifferentiated embryonic stem cells, from embryos isolated from timed matings, and from other murine tissues. The Northern blots were probed with the 1.4 kb Nsdhl EST 605654 or a 780 bp human GAPDH cDNA (control) and exposed under X-ray film. RT-PCR was also performed on bone/cartilage dissected from femurs of newborn mice.

High levels of expression of Nsdhl transcript were detected in undifferentiated embryonic stem cells and mid and late gestation mouse embryos. In adult mice, expression was detected in all tissues with the highest expression seen in ovary, testis, liver, adrenal, and kidney. Expression was also observed in eye, skin and newborn bone/cartilage, tissues that are affected in surviving Bpa females.

### EXAMPLE 2

To determine the substrate of the Nsdhl gene product sterol metabolism was examined in Bpa mice.

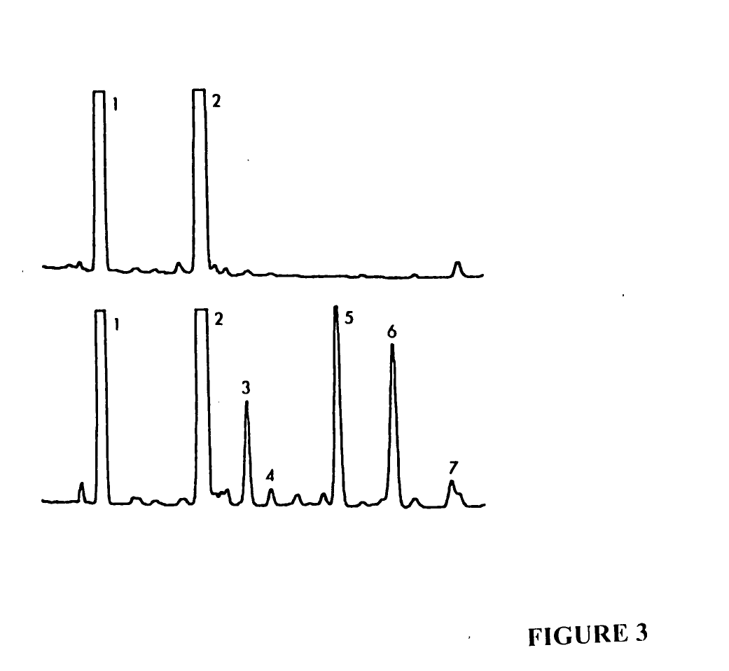

Sterol distributions in cultured skin fibroblasts or tissue samples obtained from Bpa females and control mice were analyzed by gas chromatography and selected-ion mass spectrometry as described in Kelley et al. (1999), supra except that the gas chromatographic matrix was 5% phenylmethylsilicone (HP-2, Hewlett Packard). Primary murine fibroblasts were expanded by weekly subculture (1:2) in RPMI 1640 supplemented with 15% fetal calf serum and incubated at 37° in 5% CO₂. For analysis of sterol metabolism, cells were subcultured 1:3 in T25 flasks and fed at 24 hours and 7 days with RPMI containing 15% delipidated fetal calf serum as described in Gibson et al, J. Lipid Res., 31: 515-521 (1990). After 14 days growth in delipidated medium, the cells were harvested and sterols quantitated. Tissues from female Bpa mice were analyzed for sterol content in the same manner as skin fibroblasts following initial homogenization of the tissue in 1 ml of sterol saponification solution in a ground glass homogenizer. Results of the analyses are presented in Table 1 below and in FIG. 3 which shows gas chromatographic flame ionization profiles of the sterol extracts of the normal mouse fibroblasts (upper tracing) and the Bpa fibroblasts (lower tracing). In the figure, the ordinates are detector response and the abscissae are elution time. The identified compounds are: (1) internal standard (epicoprostanol); (2) cholesterol; (3) desmosterol; and (4) lathosterol. Tentatively identified compounds include: (5) 4-methylcholest-8(9)-en-3β-ol; (6) 4-methylcholesta-8(9),24-dien-3β-ol; and (7) 4,4′-dimethylcholest-8(9)-en-3β-ol.

TABLE 1
Sterol Analysis of Cultured Skin Fibroblasts from Bpa Mice

| | Female Bpa | Female Bpa | Female Bpa | Male Mouse |
|---|---|---|---|---|
| | Culture 1 | Culture 2 | Mean | Controls (4) |
| Sterol | % Sterols | % Sterols | % Sterols | % Sterols |
| Cholesterol | 79.3 | 63.4 | 71.4 | 100.0 |
| Desmosterol | 3.3 | 5.8 | 4.6 | <0.1 |
| Lathosterol | 0.6 | 3.7 | 2.1 | <0.1 |
| 4-Methylcholest-8(9)-en-3β-ol | 7.9 | 3.9 | 5.9 | <0.1 |
| 4-Methylcholesta-8(9).24,dien- | 6.8 | 17.8 | 12.3 | <0.1 |
| 3β-ol | | | | |
| 4,4-Diemthylcholest-8(9)-en- | 1.1 | 1.0 | 1.1 | <0.1 |
| 3β-ol | | | | |

Cultured fibroblasts from an affected adult Bpa female accumulated large amounts of two C28 sterols and a smaller amount of a C29 sterol (Table 1 and FIG. 2). Similar abnormal accumulations in kidney and liver tissue from Bpa females, ages 6 days, 18 days, and 3 months were also found (data not shown). Although the exact isomeric structures of the accumulated C28 sterols remain to be determined, their fragmentation patterns and retention times are consistent with C-4 methylsterols having double bonds in, respectively, the 8(9) and 8(9), 24 positions. The C29 sterol as was tentatively identified as 4,4′-dimethylcholest-8(9)-en-3β-ol. The abnormal sterol profile is consistent with the function of Nsdhl as a C-3 sterol dehydrogenase involved in the complex series of reactions that result in the sequential removal of the two C-4 methyl groups from the sterol backbone of the cholesterol precursor lanosterol.

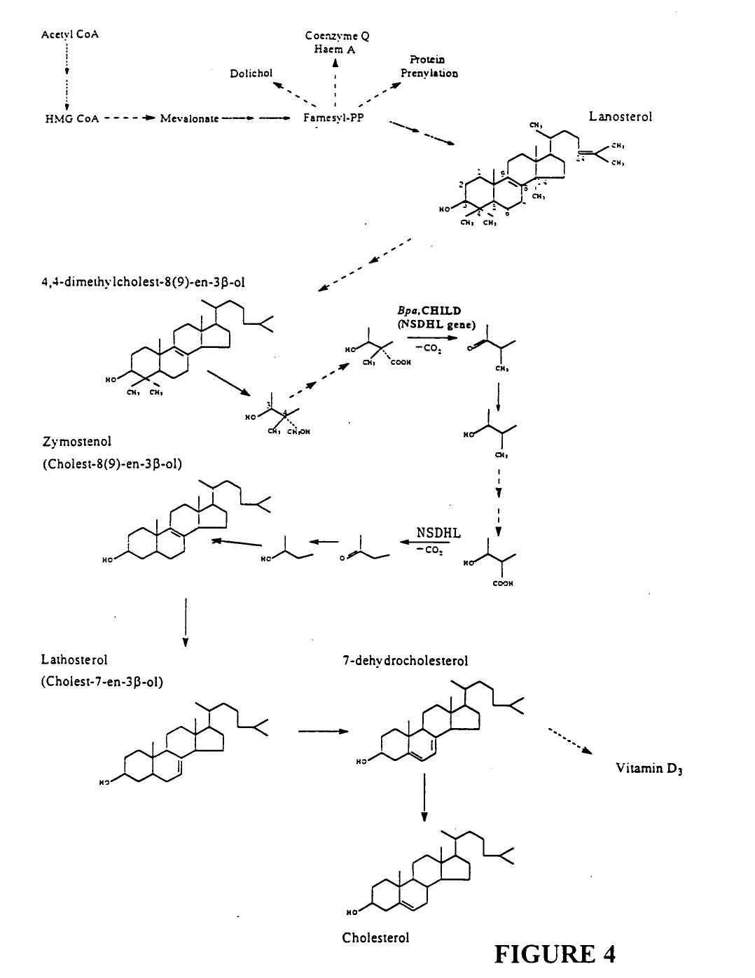

FIG. 1 is a schematic representation of the enzymatic pathway for conversion of lanosterol to cholesterol in which sterol intermediates that are increased in Bpa mice are shown in bold type and underlined. The saturation of the C-24 methyl groups may occasionally precede that of the C-24 double bond or may occur at different points in the pathway, and removal of the C-4 methyl groups may occasionally precede that of the C-14 methyl group (not shown). FIG. 4 depicts additional steps in the cholesterol biosynthetic pathway that could be affected by the accumulation of sterol intermediates.

### EXAMPLE 3

Mutations in the human NSDHL gene have been identified in patients with CHILD syndrome (congenital hemidysplasia, ichthyosiform erythroderma and limb defects), a rare X-linked dominant genodermatosis characterized by unilateral ichthyosiform skin lesions, ipsilateral anomalies of limbs and internal organs, and punctate calcifications of the epiphyses and other cartilaginous structures on the affected side.

CHILD Syndrome

In CHILD syndrome patients, there is unilateral distribution of ichthyotic skin at birth, with a sharp line of demarcation between normal and abnormal skin at the midline of the trunk. In most patients, large areas of skin are diffusely abnormal, often on the trunk, but also on the limbs. However, some skin lesions on the affected side may follow lines of Blaschko with interspersed streaks of normal skin. The skin of the face is usually spared, although scalp alopecia may be present. The skin lesions are usually most severe at or shortly after birth and often improve spontaneously, but some patients have persistent patches of involved skin that do not resolve and respond poorly to therapy. Clinically, the skin appears scaly and erythematous. The lesions may be pruritic. Skin lesions in NSDHL deficiency CHILD syndrome are characterized by waxy yellow plaques with a propensity for skin folds. Histologically, the epidermis shows orthohyperkeratosis, parakeratosis, and marked acanthosis with inflammatory infiltrates of lymphocytes, histiocytes and neutrophils. Accumulations of neutrophils may form in the stratum corneum. Verruciforn xanthoma, characterized by foamy, lipid-filled histiocytes in the dermal papillae, has also been observed in several reported cases of CHILD syndrome. New patches of involved skin, on the same or on the contralateral side, can develop. Lesions may appear and disappear spontaneously, and usually do not respond well to most therapeutic trials of emollients, topical steroids, etc.

Patients with skin disorders such as ichthyosiform nevus, ichthyosis, inflammatory linear verrucous epidermal nevus (ILVEN), linear epidermal nevus, epidermal nevus, inflammatory epidermal nevus, epidermal hyperplasia, psoriasiform erythroderma and psoriasiform epidermal nevus, which are clinically very similar to the skin abnormality seen in NSDHL deficiency CHILD syndrome, may have NSDHL deficiency or other sterol biosynthetic defects at the same enzymatic step. ILVEN may represent the mild end of the spectrum of the same defect as that in CHILD syndrome.

Limb defects may range from mild hypoplasia of digits to agenesis of an entire limb. Punctate calcifications of cartilaginous structures are often observed in newborns or young infants, but usually resolve over time. Finger and toenails may be dysplastic. In addition to the skeletal and cutaneous manifestations, patients may have ipsilateral visceral anomalies, including brain, renal, and cardiac defects. The right side is involved more frequently than the left. If the left side is affected, there is a higher risk for a serious or lethal cardiac defect.

Identification of Mutations

A distinct splicing mutation was identified in two unrelated CHILD syndrome patients. The mutation IVS7-2 A to C, alters the splice acceptor site at the beginning of exon 8 of the NSDHL gene. The presence of the heterozygous mutation was confirmed by sequencing PCR products. The mutation was also found to be present in one of the patients' asymptomatic mother by direct sequencing. Confirmation of the mutation was performed by RT-PCR performed as described in Levin et al., Genome Res., 6: 465-477 (1996) on patient lymphoblast DNA using the primers shown below that amplify between exon 7 and exon 8 of the DNA sequence.

| | | |
|---|---|---|
| Forward | 5′ ACGTGGTCCATGGACACA 3′ | (SEQ ID NO: 27) |
| Reverse | 5′ CATCCATGGTCACTAGTGGC 3′ | (SEQ ID NO: 28) |

PCR conditions were 94° C., 7 minutes; 94° C., 30 seconds; 55° C., 30 seconds; 72° C., 30 seconds; then forty cycles of 72° C., 7 minutes and 4° C. The RT-PCR product was excised from a gel and directly sequenced. It indicated that the mutation results in abnormal splicing and the in frame deletion of the first fifty-six amino acids in exon 8 of the protein.

Other mutations have been identified in the NSDHL gene of CHILD patients and are described in König et al., Am. J. Med. Genet., 90: 339-346 (2000), which is incorporated by reference herein.

Sterol Analysis

Sterol analysis of the serum and/or skin fibroblasts of the patients and mother demonstrated the accumulation of 4-methyl sterols consistent with the mutation in the NSDHL gene.

### EXAMPLE 4

Abnormal sterol accumulation consistent with defects in the genes encoding enzymes in the NSDHL step was also observed in psoriasis patients.

Psoriasis

Psoriasis is a chronic papulosquamous disorder that undergoies spontaneous remissions and exacerbations. In psoriasis, skin lesions are sharply demarcated with distinct borders. The lesions of psoriasis demonstrate a variety of morphologic types and areas of distribution on the body. The surface of the lesions is covered with silvery scales, and under the scale, there is glossy, homogeneous erythema. Nails are frequently involved in psoriasis; the abnormalities can include nail pits, brownish or yellowish discoloration beneath the nail plate, or severe onychodystrophy. Histopathology of psoriasis is characterized by thickened (3-5× normal) epidermis (acanthosis), parakeratosis, and a lymphohistiocytic infiltrate in the papillary dermis. The dermal papillae become thin and elongated, and contain tortuous blood vessels in an edematous stroma. Psoriasis, its diagnosis, and its treatment are discussed in Abel, Psoriasis 8/97, 2. Dermatology III, pp. Psoriasis-1 to Psoriasis-14, in Scientific American Medicine, Scientific American, Inc., New York, N.Y. (1973-2000), which is incorporated by reference herein.

Differences between psoriasis and the NSDHL deficiency skin lesions include an apparent difference in the clinical appearance of the skin scales, in that the scales in psoriasis are silvery and those of NSDHL deficiency are waxy and yellow, and the lack of verruciform xanthomatous changes in the dermis in psoriasis on biopsy. However, in psoriasis and other ichthyoses, such as harlequin ichthyosis, lipid vacoules are seen in lesional keratinocytes ultrastructurally. Keratinocytes may be vacoulated in CHILD syndrome as well.

Sterol Analysis

To determine if common psoriasis is associated with the same abnormalities of cholesterol biosynthesis that are characteristic of both human NSDHL- and murine Nsdhl-deficiency, sterols were quantified in the scales of human psoriatic skin and from forearm skin of human adult controls. Presented in Table 2 below are representative data summarizing sterol levels in the skin of three adult psoriasis samples, three normal adult controls, and one of the CHILD syndrome patients discussed in Example 3, wherein all values are percent of total sterols

TABLE 2
Sterol Analysis in Psoriasis

| | Cho- | | | |
|---|---|---|---|---|
| | lesterol | Squalene | 4-Methylsterol-1 | 4-Methylsterol-2 |
| Psoriasis-1 | 90 | 0.9 | 0.3 | 1.2 |
| Psoriasis-2 | 88.6 | 0.6 | 0.2 | 2.0 |
| Psoriasis-3 | 89.4 | 0.5 | 1.0 | 3.7 |
| Control 1 | 42.0 | 42.0 | <0.1 | <0.1 |
| Control 2 | 57.0 | 37.1 | <0.1 | <0.1 |
| Control 3 | 56.0 | 36.9 | <0.1 | <0.1 |
| CHILD | 96 | 0.4 | 1.3 | 0.3 |

All psoriasis samples tested had increased levels of the same two 4-methylsterols that are most prominent in Nsdhl-deficient mouse plasma, tissues, and cultured cells: 4-methylcholest-8(9)-en-3beta-ol (methylsterol-1) and 4-methylcholesta-8(9),24-dien-3beta-ol (methylsterol-2). These are the sterols that are predicted herein to be increased because of the deficient activity of the NSDHL-encoded 3β-HSD of the sterol-4-demethylase complex. Only trace amounts of these compounds are present in normal skin. Another important characteristic of the skin of the human psoriasis is the near absence of squalene, a isoprenoid precursor of lanosterol and one of the most abundant lipids in normal skin.

Essentially the same abnormal pattern of sterols was found in the skin of the CHILD syndrome patient with the established NSDHL mutation described in Example 3 above. One difference between the sterol profile of CHILD syndrome skin and that of psoriasis skin is the reversed ratio of the two 4-methylsterols. Nevertheless, the similarity of the two sterol profiles indicates that dysfunction of the sterol-4-demethylase enzyme complex is a characteristic of human psoriasis in the patients examined.

Diagnostic methods based on the analysis of genes encoding enzymes participating in the sterol-4-demethylase complex or on analysis of sterol accumulation are therefore contemplated as useful to clinicians in the initial diagnosis of psoriasis and also in determining treatment course as different psoriasis patients are not necessarily responsive to the same therapies.

While the present invention has been described in terms of exemplary methods, it is understood that variations and modifications will occur to those skilled in the art. Therefore, only such limitations as appear in the claims should be placed on the invention.

We claim:

1. A method of diagnosing CHILD syndrome in a patient comprising the steps of:
(a) isolating patient NSDHL polynucleotide; and
(b) detecting a nucleotide difference between patient NSDHL polynucleotide and the wild type NSDHL gene.

2. A method of diagnosing CHILD syndrome comprising the steps of:
(a) isolating body fluid or cells from a patient; and
(b) detecting in the body fluid or cells accumulation of a sterol intermediate or metabolite thereof prior to the step of generation of cholest-8(9)-en-3β-ol in the cholesterol biosynthetic pathway.

3. The method of claim 2 wherein the sterol intermediate detected is selected from the group consisting of 4,4-dimethylcholest-8(9)-en-3β-ol, 4-methylcholest-8(9)-en-3β-ol, 4-methylcholesta-8(9),24-dien-3β-ol and metabolites thereof.

4. A method of diagnosing psoriasis comprising the steps of:
(a) isolating body fluid or cells from a patient; and
(b) detecting in the body fluid or cells accumulation of a sterol intermediate or metabolite thereof prior to the step of generation of cholest-8(9)-en-3β-ol in the cholesterol biosynthetic pathway.

5. The method of claim 4 wherein the sterol intermediate detected is selected from the group consisting of 4,4-dimethylcholest-8(9)-en-3β-ol, 4-methylcholest-8(9)-en-3β-ol, 4-methylcholesta-8(9),24-dien-3β-ol and metabolites thereof.


SEQUENCE LISTING (28 sequences)

SEQ ID NO: 1 - DNA, 1563 bp, Homo sapiens
  CDS (195)..(1313)
cgccgagctgggccaatcctcttggtggaggaagctcggctgattctcggctcacgcgggaggggagtaaagggtggcggtccgggcctggagttcagtgggtgcagcctgcttgcgagctgaggccagacaggggggcgcctacggacggaaaagaaaagttgattacaaacgggaccatattttgcttcgaaatggaaccagcagttagcgagccaatgagagaccaagtcgcacggactcatttgacagaggacactcccaaagtgaatgctgacatagaaaaggttaaccagaatcaggccaagagatgcacagtgatcggtggctctggattcctggggcagcacatggtggagcagttgctggcaagaggatatgctgtcaatgtatttgatatccagcaagggtttgataatccccaggtgcggttctttctgggtgacctctgcagccgacaggatctgtacccagctctgaaaggtgtaaacacagttttccactgtgcgtcacccccaccatccagtaacaacaaggagctcttttatagagtgaattacattggcaccaagaatgtcattgaaacttgcaaagaggctggggttcagaaactcattttaaccagcagtgccagtgtcatctttgagggcgtcgatatcaagaatggaactgaagaccttccctatgccatgaaacccattgactactacacagagactaagatcttacaggagagggcagttctgggcgccaacgatcctgagaagaatttcttaaccacagccatccgccctcatggcattttcggcccaagggacccgcagttggtacccatcctcatcgaggcagccaggaacggcaagatgaagttcgtgattggaaatgggaagaacttggtggacttcacctttgtggagaacgtggtccatggacacatcctggcggcagagcagctctcccgagactcgacactgggtgggaaggcatttcacatcaccaatgatgagcccatccctttctggacattcctgtctcgcatcctgacaggcctcaattatgaggcccccaagtaccacatcccctactgggtggcctactacctggccctcctgctatccctgctggtgatggtgatcagtcctgtcatccagctgcagcccaccttcacacccatgcgggtcgcactggctggcacattccactactacagctgcgagagagccaaaaaggccatgggctaccagccactagtgaccatggatgatgctatggagaggaccgtgcagagctttcgccacctgcggagggtcaagtgagggacactggaggctgggctctctcgacacgttgctcagccagtcactccttcccctgtggattgatgaaataacatcctttgaatgagtttgctctgagcctgtgactccttctgctaggcagagagcgcaccctactctttccgtgacgatgagggcggcaaaaacagacatttcttccttcatggaactggatttggatttcttgaagcaggcagcttcatattataccgatttgttctctgtc

SEQ ID NO: 2 - protein, 373 aa, Homo sapiens
MEPAVSEPMRDQVARTHLTEDTPKVNADIEKVNQNQAKRCTVIGGSGFLGQHMVEQLLARGYAVNVFDIQQGFDNPQVRFFLGDLCSRQDLYPALKGVNTVFHCASPPPSSNNKELFYRVNYIGTKNVIETCKEAGVQKLILTSSASVIFEGVDIKNGTEDLPYAMKPIDYYTETKILQERAVLGANDPEKNFLTTAIRPHGIFGPRDPQLVPILIEAARNGKMKFVIGNGKNLVDFTFVENVVHGHILAAEQLSRDSTLGGKAFHITNDEPIPFWTFLSRILTGLNYEAPKYHIPYWVAYYLALLLSLLVMVISPVIQLQPTFTPMRVALAGTFHYYSCERAKKAMGYQPLVTMDDAMERTVQSFRHLRRVK

SEQ ID NO: 3 - DNA, 2218 bp, Mus musculus
  CDS (216)..(1301)
gagcgtcaattggttgcgccagagccaagctagaccaatcaacattatgaaggaagctctgctgattgtgggctcatatctatcagtagaaaagggtggcgggtgttcagccagacttctctggttgccggttgtctgcaagctgaggtcgatcatttgagtgtctaaaccgggaagaagagttgattgcaaacgaaaccatactttgagccataatggaacaagctgttcatggtgaatcaaagcgaggccaggtcacaggaacacatttgacaaatgacatttccaaagctaagaagtgcacagtgattggaggctctgggttcctggggcagcacatggtggagcagttgctggagcgaggctatactgtcaatgtatttgatatccaccaaggctttgataacccccgggtgcagttctttataggcgacctgtgcaaccaacaggacctgtacccagctctcaaaggtgtaagcacagttttccactgcgcgtcccctccgccgtacagtaacaacaaggagctcttttatagagtgaatttcattggcaccaagactgtcattgaaacctgcagagaggccggagttcagaaactcattttaaccagcagtgccagtgttgtctttgagggtgttgacataaaaaatggaactgaagacctcccttacgccatgaagcctattgactattacacagagaccaagatcttgcaggagagagcagtactggatgccaacgaccctaagaagaattttttaaccgcagccattcgtcctcatggcattttcggcccaagggacccccagttggtcccaatcctaattgatgcagctagaaagggcaaaatgaagttcatgattggaaatggggaaaacctggtggacttcaccttcgtggagaatgtggttcatggacacatcttagccgctgagcacctctcccaagatgcagctctaggtggaaaggcatttcacatcaccaacgatgaaccaatccctttctggacgttcctgtcccgcattctgacaggcctcaattatgaggcccctaagtaccacatcccctactggatggcctattaccttgctttcctgctatctctactggtgatggtggtcagccctctcatccaaatccagccaacctttacaccaattcgagtggcattggctggaacattccactattacagttgtgaaaaagccaaaaagctctttgggtaccggccactggtcaccatggatgaagctgtggaaaggactgtgcagagtttccaccacctgcggaaggacaagtgaagttccccagtgccctccccagtaactttctcctctctgttctaaagaacgcatatccttaactgagtttctcttgacctgtgtgtcccttgctggttagatggtagcatacctgacaccctccatgacattggaggtgacaacaggagacatttctcccaggatagcattgaacttctcaggcagagtcataatctactgcttgggtctttttctctcccccacacccctacttctgtctcctgggttcattatcagaaagacagcactaaagtgaagtctttatctggggtcttaaaaattgaagcaaaaccagaaattgtaaacacacagtaagccttcagacatacattttatatgatcacagtacaatagctcaaagtattgatgaatgtaatccccaatccttaaggataaatccactgctggttccttgcccctcacatactgtctaggtctctttcaaagatggttgcagtgtctgcctctattgtttttccataaatcatttcaatttatccacagactctggtgtggtctttattgctcagtgatggtgcaatgcaatggtacagtgtgtgctgtgttagctggtcagctcctaataaacaggagatgatggctgcttggcaataagccaaaaagagtggtctccttcatcgacatgatgtttgagtagaaactcctccgtgagatggggcagaatgggttacagacatctttgcagctcatttcacagaacattattaggccactttccaaagagtacaggccactaacaatccagccatttctgttctctgaactcattgaaatgtattcttgaagaattctaagttcttcttcatcactgttcaacagtaagatgattgggggaaaataacaaagaaataaaagccatacagtttgtgttg

SEQ ID NO: 4 - protein, 362 aa, Mus musculus
MEQAVHGESKRGQVTGTHLTNDISKAKKCTVIGGSGFLGQHMVEQLLERGYTVNVFDIHQGFDNPRVQFFIGDLCNQQDLYPALKGVSTVFHCASPPPYSNNKELFYRVNFIGTKTVIETCREAGVQKLILTSSASVVFEGVDIKNGTEDLPYAMKPIDYYTETKILQERAVLDANDPKKNFLTAAIRPHGIFGPRDPQLVPILIDAARKGKMKFMIGNGENLVDFTFVENVVHGHILAAEHLSQDAALGGKAFHITNDEPIPFWTFLSRILTGLNYEAPKYHIPYWMAYYLAFLLSLLVMVVSPLIQIQPTFTPIRVALAGTFHYYSCEKAKKLFGYRPLVTMDEAVERTVQSFHHLRKDK

SEQ ID NO: 5 - DNA, 1751 bp, Homo sapiens
  CDS (27)..(908)
gcgagatgactgcagagatttgaaaaatggcaacaaatgaaagtgtcagcatctttagttcagcatccttggctgtggaatatgtagattcacttttacctgagaatcctctgcaagaaccatttaaaaatgcttggaactatatgttgaataattatacaaagttccagattgcaacatggggatcccttatagttcatgaagccctttatttcttattctgtttacctggatttttatttcaatttataccttatatgaaaaaatacaaaattcaaaaggataagccagagacatgggaaaaccaatggaagtgtttcaaagttcttctctttaatcacttctgtatccagctgcctttgatttgtggaacctattattttacagagtatttcaatattccttatgattgggaaagaatgccaagatggtattttcttttggcaagatgctttggttgtgcagtcattgaagatacttggcactattttctgcatagactcttacaccacaaaagaatatacaagtatattcataaagttcatcatgagtttcaggctccatttggaatggaagctgaatatgcacatcctttggagactctaattcttggaactggatttttcattggaatcgtgcttttgtgtgatcatgtaattcttctttgggcatgggtgaccattcgtttattagaaactattgatgtccatagtggttatgatattcctctcaaccctttaaatctgatccctttctatgctggttctcggcatcatgatttccaccacatgaacttcattggaaactatgcttcaacatttacatggtgggatcgaatttttggaacagactctcagtataatgcctataatgaaaagaggaagaagtttgagaaaaagactgaataaatatctcacgtaaaccttcctgaaagataaacgttttcctgaattcagaaactagtagctaacattgcttctggagagcagaaataagcatgtcttctggctactaagtgataaaaagaacattaacaacctttaattaccttcctagtgggaactttttctactttacctacaagttctatatatgtagaaatgaataaatatatatttaagtacagttttcatgaggaagttttaaaagaccatgttcctaagcttccaagaaggttttggatactagaagtattaatctatggcttttctcccagtaaaaccataggcctgaagttcacattgggtctttaaatcttttagatatatactggtcatttcagaaaattcttcatagtggtattggccttatatttaactttttttttattttttttttgagacaaagccacactctgtctccttgtctggagtgtggtggcacagtctcagctcactgcaacctctgcctcccagttcaagcaattcttctgcctcagcctcccaagtagctgggattacaggcacccgccaccacgcccagctaatttttgtatttttgtagagatggggtttctcgatgttggccaggctggtctcaaacttctgacctcaagtgatctgcccaccttggcctcccaaagtgctgggattacaggtgtaagccactgcgcccggcctttttaactttaaacatgttttagaattcacctaaagatcaaaatatcatggattgaacctcatcaattgatagcagtgagtgactgaagcttccaaatcaagaaaagccggcaccaagaacttccattctaa

SEQ ID NO: 6 - protein, 293 aa, Homo sapiens
MATNESVSIFSSASLAVEYVDSLLPENPLQEPFKNAWNYMLNNYTKFQIATWGSLIVHEALYFLFCLPGFLFQFIPYMKKYKIQKDKPETWENQWKCFKVLLFNHFCIQLPLICGTYYFTEYFNIPYDWERMPRWYFLLARCFGCAVIEDTWHYFLHRLLHHKRIYKYIHKVHHEFQAPFGMEAEYAHPLETLILGTGFFIGIVLLCDHVILLWAWVTIRLLETIDVHSGYDIPLNPLNLIPFYAGSRHHDFHHMNFIGNYASTFTWWDRIFGTDSQYNAYNEKRKKFEKKTE

SEQ ID NO: 7 - DNA, 20 bp, Artificial Sequence
  Description of Artificial Sequence CH-F primer
aaagactggtgcgctaaagc

SEQ ID NO: 8 - DNA, 20 bp, Artificial Sequence
  Description of Artificial Sequence CH-R primer
cgagcttcctccaccaagag

SEQ ID NO: 9 - DNA, 20 bp, Artificial Sequence
  Description of Artificial Sequence hH105-1F primer
ccccgtctttattgggcaag

SEQ ID NO: 10 - DNA, 21 bp, Artificial Sequence
  Description of Artificial Sequence hH105-1R primer
actgcccagtcgctgacacag

SEQ ID NO: 11 - DNA, 22 bp, Artificial Sequence
  Description of Artificial Sequence hH105-2F primer
ggcatctgcccaaaacactaac

SEQ ID NO: 12 - DNA, 22 bp, Artificial Sequence
  Description of Artificial Sequence hH105-2R primer
ccacaggtaaatagtatcagcc

SEQ ID NO: 13 - DNA, 20 bp, Artificial Sequence
  Description of Artificial Sequence hH105-3F primer
ttccagtcctcactaccctg

SEQ ID NO: 14 - DNA, 20 bp, Artificial Sequence
  Description of Artificial Sequence hH105-3R primer
agtatcgtggtttcccttcg

SEQ ID NO: 15 - DNA, 20 bp, Artificial Sequence
  Description of Artificial Sequence hH105-4F primer
tgccattgacctgtcaaagc

SEQ ID NO: 16 - DNA, 20 bp, Artificial Sequence
  Description of Artificial Sequence hH105-4R primer
cccttagaaagggccatcac

SEQ ID NO: 17 - DNA, 22 bp, Artificial Sequence
  Description of Artificial Sequence hH105-5F primer
ggatcatgcactgtttgaattg

SEQ ID NO: 18 - DNA, 20 bp, Artificial Sequence
  Description of Artificial Sequence hH105-5R primer
ggattctaaacccttcagtc

SEQ ID NO: 19 - DNA, 20 bp, Artificial Sequence
  Description of Artificial Sequence hH105-6F primer
ctaggaatttgcaatggacg

SEQ ID NO: 20 - DNA, 20 bp, Artificial Sequence
  Description of Artificial Sequence hH105-6R primer
tgaatgcgagcatggaccag

SEQ ID NO: 21 - DNA, 20 bp, Artificial Sequence
  Description of Artificial Sequence hH105-7F primer
aagacttgggagtggccctg

SEQ ID NO: 22 - DNA, 20 bp, Artificial Sequence
  Description of Artificial Sequence hH105-7R primer
aggcaaggagaagaaacccg

SEQ ID NO: 23 - DNA, 20 bp, Artificial Sequence
  Description of Artificial Sequence hH105-8F1 primer
ttcaactttgggcaggtggg

SEQ ID NO: 24 - DNA, 20 bp, Artificial Sequence
  Description of Artificial Sequence hH105-8R1 primer
ctccatagcatcatccatgg

SEQ ID NO: 25 - DNA, 20 bp, Artificial Sequence
  Description of Artificial Sequence hH105-8F2 primer
cattccactactacagctgc

SEQ ID NO: 26 - DNA, 20 bp, Artificial Sequence
  Description of Artificial Sequence hH105-8R2 primer
tgtataaaccagaagagggg

SEQ ID NO: 27 - DNA, 18 bp, Artificial Sequence
  Description of Artificial Sequence forward primer
acgtggtccatggacaca

SEQ ID NO: 28 - DNA, 20 bp, Artificial Sequence
  Description of Artificial Sequence reverse primer
catccatggtcactagtggc